(19) United States
(12) Patent Application Publication
(10) Pub. No.: US 2020/0340983 A1
(43) Pub. Date: Oct. 29, 2020

(54) METHOD FOR IMMOBILIZING LIGAND HAVING AMINO GROUP

(71) Applicant: KANEKA CORPORATION, Osaka (JP)

(72) Inventor: Takuma Suzuki, Osaka (JP)

(73) Assignee: KANEKA CORPORATION, Osaka (JP)

(21) Appl. No.: 16/926,972

(22) Filed: Jul. 13, 2020

Related U.S. Application Data
(63) Continuation of application No. PCT/JP2019/000016, filed on Jan. 4, 2019.

(30) Foreign Application Priority Data
Jan. 12, 2018 (JP) 2018-003100

(51) Int. Cl. G01N 33/543; B01J 20/24; B01J 20/32; B01J 20/28

(52) U.S. Cl. CPC G01N 33/543; B01J 20/24; B01J 20/3212; B01J 20/28033; B01J 20/3248; B01J 20/28016; B01J 20/28042; B01J 20/3219

(57) ABSTRACT

A method is provided for strongly immobilizing a ligand by inactivating an excess formyl group. Methods are also provided for immobilizing a ligand on a formyl group-containing insoluble base material, where the ligand has a specific affinity for a target compound and also has an amino group. The methods comprise the steps of mixing the ligand with the formyl group-containing insoluble base material to form an imine, and reducing the imine by using two or more kinds of reducing agents.

### TECHNICAL FIELD

One or more embodiments of the present disclosure relate to a method for efficiently immobilizing a ligand having an amino group on a formyl group-containing insoluble base material.

### BACKGROUND

A usability of a bioactive substance such as a peptide having a specific affinity for a specific compound and a substrate of an enzyme is enhanced by immobilizing the bioactive substance on an insoluble base material, since a substance which interacts with the immobilized bioactive substance can be recovered and detected. For example, only a target compound can be obtained from a mixed liquid with good efficiency by affinity chromatography in which a bioactive substance specifically binding to the target compound is immobilized as a ligand on an insoluble porous particle. For example, affinity chromatography is industrially used for separating an immunoglobulin by using an immobilized protein or used for separating an antigen by using an immobilized antibody.

It is very important for an industrial application to immobilize a ligand on an insoluble base material by a strong covalent bond in order to reduce a leakage of the immobilized ligand. In addition, a condition of an immobilized ligand is also important, and it is preferred that a ligand is immobilized while maintaining the activity thereof.

As a method for immobilizing a ligand on an insoluble base material, for example, a method in which a ligand is immobilized on an insoluble base material by reductive amination reaction has been developed. Specifically, a formyl group is introduced on an insoluble base material, the formyl group is reacted with a ligand having an amino group to be imine, and the imino group is reduced to be a stable amine (Patent document 1).

In addition, Patent document 2 discloses a method for remarkably reducing a leakage amount of a ligand by using the specific reducing agent in the same reaction.

### PATENT DOCUMENT

Patent Document 1: JP 2015-110224 A
Patent Document 2: WO 2017/034024

The inventors of the present disclosure however found that there is room for improvement on a leakage amount of a ligand in the method described in Patent document 1 and there is room for improvement on an inactivation of an excess formyl group in the method described in Patent document 2.

### SUMMARY

One or more embodiments of the present disclosure is to provide an immobilization method in which the method may be excellent in inactivating an excess formyl group and in reducing a leakage amount of a ligand.

The inventors of the present disclosure conducted extensive studies. As a result, the inventors completed the present disclosure by finding that a ligand can be immobilized on an insoluble base material more surely and an excess formyl group can be reduced by using two or more kinds of reducing agents.

Some of the embodiments of the present disclosure can be described by the following [1] to [9].

[1] A method for immobilizing a ligand having an amino group on a formyl group-containing insoluble base material, comprising the steps of:

mixing the ligand with the formyl group-containing insoluble base material to form an imine, and reducing the imine by using two or more kinds of reducing agents.

[2] The method according to the above [1], wherein the two or more kinds of reducing agents are separately added to reduce the imine.

[3] The method according to the above [1] or [2], wherein the imine is reduced by using a borane complex as the reducing agent and then a different reducing agent, the borane complex has a Lewis base ligand, and pKa of the Lewis base ligand is 6.5 or less.

[4] The method according to the above [3], wherein the Lewis base ligand having pKa of 6.5 or less is a nitrogen-containing heteroaryl compound.

[5] The method according to any one of the above [1] to [4], wherein the ligand is a peptide.

[6] The method according to the above [5], wherein the peptide can specifically bind to an antibody.

[7] The method according to any one of the above [1] to [6], wherein the formyl group-containing insoluble base material is composed of at least one selected from the group consisting of a polysaccharide, a synthetic polymer and a glass.

[8] The method according to any one of the above [1] to [7], wherein a form of the formyl group-containing insoluble base material is at least one selected from the group consisting of a porous particle, a monolith and a porous membrane.

[9] A method for purifying a target compound, comprising the steps of:
immobilizing the ligand on the formyl group-containing insoluble base material to produce an adsorbent by the method according to any one of the above [1] to [8],
contacting a mixed liquid containing the target compound with the adsorbent to adsorb the target compound on the adsorbent, and
separating the target compound adsorbed on the adsorbent from the adsorbent.

According to one or more embodiments of the present disclosure, a leakage amount of a ligand can be remarkably reduced and simultaneously an excess formyl group can be sufficiently inactivated. One or more of the embodiments of the present disclosure is therefore industrially excellent as a specific adsorbent to obtain a highly pure target compound with reduced amount of mixed impurity can be produced by one or more embodiments of the present disclosed method.

### DETAILED DESCRIPTION

Hereinafter, one or more embodiments of the present disclosure is described, but the present disclosure is not restricted to the following embodiments.

1. Step to Introduce Formyl Group on Insoluble Base Material

When a formyl group-containing insoluble base material is available, for example, when a formyl group-containing insoluble base material is commercially supplied, the present step is not needed to be performed. On the one hand, when a formyl group-containing insoluble base material is not available, a formyl group is introduced on an insoluble base material in accordance with a publically known method.

An insoluble base material is not particularly restricted as long as the base material is insoluble to a solvent of a mixed liquid containing a target compound, such as water, and the target compound is adsorbed onto the base material. An example of the insoluble base material includes a porous particle used as a filler for chromatography, a biosensor of an analyzer to detect a target compound, a monolith used for separating, recovering or analyzing a target compound, a porous membrane used for separating a target compound to be recovered or for removing a contaminant, and a chip such as a protein microarray. An example of a biosensor of an analyzer includes a sensor chip of an analyzer using surface plasmon resonance or biolayer interferometry.

A raw material of the insoluble base material is not particularly restricted as long as the raw material is insoluble to a solvent of a mixed liquid containing a target compound, such as water, and is exemplified by a polysaccharide such as cellulose, agarose, dextran, starch, pullulan, chitosan and chitin; a synthetic polymer such as poly(meth)acrylic acid, poly(meth)acrylate ester, polyacrylamide and polyvinyl alcohol; and a glass such as silica glass, borosilicate glass, optical glass and soda glass. In addition, the surface of a base material composed of a synthetic polymer which does not have a functional group, such as polystyrene and styrene-divinylbenzene copolymer, may be coated with a polymer material having a reactive functional group such as a hydroxy group. An example of such a polymer material for coating includes a graft copolymer such as a copolymer of hydroxyethyl methacrylate or a monomer having a polyethylene oxide chain and other polymerizable monomer having a reactive functional group. A polysaccharide and a polyvinyl alcohol are preferably used among the above-described raw materials, since an active group can be easily introduced on the surface of the base material.

An example of an insoluble base material form includes a porous particle, a monolith and a porous membrane.

A size of a porous particle as the insoluble base material may be appropriately adjusted, and a volume average particle diameter thereof is preferably adjusted to 20 μm or more and 1000 μm or less. When a column is filled with the porous particle having a volume average particle diameter of 20 μm or more, back pressure can be reduced to low. On the one hand, when the volume average particle diameter is 1000 μm or less, the surface area is large and an amount of an adsorbed target compound becomes large. The volume average particle diameter is more preferably 30 μm or more, more preferably 40 μm or more, even more preferably 50 μm or more, and more preferably 250 μm or less, more preferably 125 μm or less, more preferably 100 μm or less, even more preferably 85 μm or less. The volume average particle diameter of the porous particle can be determined by measuring diameters of 100 porous particles selected at random. Each porous particle diameter can be measured by taking a microscope photograph of each porous particle, saving the electronic data of the microscope photograph, and using a particle size measurement software such as “Image-Pro Plus” manufactured by Media Cybernetics, Inc. The porous particle is preferably crosslinked by using a polyfunctional compound in accordance with an ordinary method in order to increase the strength.

A monolith is a kind of a porous continuous structure and a spongelike structure monolithically composed of a skeleton to support the structure and a hole. A monolith is excellent in mass transfer property and pressure-flow velocity property. An adsorption efficiency and separation efficiency of a target compound, liquid permeability and detection sensitivity can be improved by controlling a pore size and a skeleton size thereof. It can be judged that a structure is continuously porous by confirming similar pores in different cross-sections of the structure using a scanning electron microscope or the like.

An example of a porous membrane includes a membrane having a structure such as flat membrane, hollow fiber and depth filter.

A pore diameter of the insoluble base material having a pore, such as a monolith and a porous membrane, may be adjusted depending on a target compound to be adsorbed or a flow velocity, and may be adjusted to, for example, 1 nm or more and 10 μm or less. For example, when a target compound is an antibody or an antibody fragment, a pore diameter is particularly preferably adjusted to 10 nm or more and 300 nm or less.

The insoluble base material may be produced from a raw material by a publicly known method. For example, a porous particle may be produced by dispersing a solution or a dispersion of a raw material polymer in a fat and oil or the like to be a liquid droplet and contacting the mixture with a solvent miscible in the solvent of the solution or the dispersion, such as alcohol and aqueous alcohol.

A formyl group may be introduced on the insoluble base material by utilizing a functional group of a raw material or a coating material of the insoluble base material. For example, a polysaccharide as a raw material has many hydroxy groups. An epoxy group can be introduced by reacting a halohydrin such as epichlorohydrin with the hydroxy group. Alternatively, when a polyepoxide compound is used as a crosslinking agent, an unreacted epoxy group may remain. A ring of an epoxy group is easily opened by an acidic aqueous solution or a basic aqueous solution to be a 1,2-diol group. Such a 1,2-diol group can be oxidized by an oxidizing agent to be a formyl group.

An example of an oxidizing agent to oxidize a hydroxy group to be a formyl group includes periodic acid and a periodate salt. An example of a periodate salt includes sodium periodate and potassium periodate.

An amount of a formyl group in the formyl group-containing insoluble base material is not particularly restricted and is preferably 0.5 μmol or more and 100 μmol or less per 1 mL of the formyl group-containing insoluble base material. When the amount of a formyl group per 1 mL of the formyl group-containing insoluble base material is 0.5 μmol or more, a ligand can be efficiently immobilized and an amount of an adsorbed target compound may become large in the case where the base material is used for producing an adsorbent. In addition, when the amount of a formyl group per 1 mL of the formyl group-containing insoluble base material is 100 μmol or less, an amount of an adsorbed target compound may amazingly become large, though the reason for that is unknown. When a formyl group is introduced by using periodic acid and/or a periodate salt and the amount of a formyl group per 1 mL of the formyl group-containing insoluble base material is 100 μmol or less, the strength of the formyl group-containing insoluble base material may preferably become high. The amount of a formyl group per 1 mL of the formyl group-containing insoluble base material is more preferably 1 μmol or more, even more preferably 1.5 μmol or more, even more preferably 2 μmol or more, and more preferably 75 μmol or less, even more preferably 50 μmol or less, even more preferably 40 μmol or less. The amount of a formyl group can be controlled by, for example, a time and a temperature of a reaction to introduce a formyl group and a concentration of a formylating agent such as periodic acid and a periodate salt. A volume of the formyl group-containing insoluble base material as a base of the above-described formyl group amount or the like means a volume of a whole structure including a pore and a skeleton in the case of the monolith and the porous membrane or a tapped volume in the case of the porous particle unless otherwise specified in this disclosure. The tapped volume means a volume in the condition that a slurry containing the porous particle and a dispersing medium such as water is added into a measuring vessel and the porous particle is settled down until the volume is not further decreased with vibration of the measuring vessel.

The amount of a formyl group can be determined by adding a phenylhydrazine solution to the formyl group-containing insoluble base material, stirring the mixture at 40° C. for 1 hour, measuring absorption spectrum of a supernatant using an ultraviolet visible light spectrophotometer after the reaction, and obtaining a decreased amount of phenylhydrazine using a standard curve of phenylhydrazine.

2. Step to Introduce Amino Group to Ligand

When a ligand has an amino group, the present step is not needed. On the one hand, when a ligand does not have an amino group, an amino group is introduced.

The ligand to be bound on the insoluble base material in one or more embodiments of the present disclosure may be, for example, a substance which can selectively bind to a target compound from an aggregate of some molecules by an affinity between molecules for a target compound. The ligand has an affinity for a target compound and is exemplified by a peptide, a sugar chain, a substrate of an enzyme, and DNA. The peptide means a compound which is composed of 2 or more amino acids bound through a peptide bond and which has a specific affinity for a target compound. An example of the ligand includes a protein having a specific affinity for a target compound, such as a receptor protein which binds to a substrate compound, an antibody to an antigen, and lectin which can bind to a sugar chain, a subunit and a domain which maintain a specific affinity for a target compound, and an antibody fragment such as a Fab region.

An example of a peptide which can be used as the ligand includes an affinity ligand for an antibody. Such an affinity ligand for an antibody is exemplified by Protein A, Protein G, Protein L, Protein H, Protein D, Protein Arp, Protein FcγR, an antibody-binding synthetic ligand, and analogs thereof. Such an analog of the above affinity ligand for an antibody means a mutant which is prepared by deleting, substituting and/or adding one or more amino acids of the above-described Protein A or the like and of which affinity for a target antibody or a fragment thereof is maintained or improved in comparison with a natural Protein A or the like, or a subunit or a domain of the above-described Protein A or the like of which affinity for a target antibody or a fragment thereof is maintained in this disclosure. The number of the mutation such as deletion in the above-described mutant may be adjusted depending on the amino acids which compose an original peptide, and may be adjusted to 20 or less and preferably 10 or more and 5 or less. The mutation number may be preferably 1 or more.

When a substrate of an enzyme or a sugar chain does not have an amino group, an amino group is introduced. A skilled person can easily transform a functional group of a substrate compound and a sugar chain into an amino group or introduce an amino group utilizing a functional group. When a peptide to be a ligand does not have an amino group other than the N-terminal or does not have sufficient side chain amino group, a basic amino acid such as lysine or a derivative thereof can be introduced or substituted at an arbitrary position by genetic recombination technology or synthetic technology. When DNA or a sugar does not have an available amino group or has an insufficient amino group, an amino group can be similarly introduced.

A target compound of a ligand is not particularly restricted as long as the target compound is a target to be purified or detected and the ligand can specifically bind to the target compound in this disclosure. An example of a target compound includes an immunoglobulin G (IgG) and an immunoglobulin G derivative which bind to Protein A, Protein G, Protein L, Protein H, Protein D, Protein Arp, Protein FcγR or an antibody-binding synthetic ligand; a glycoprotein binding to lectin; plusminogen binding to lysine; biotin binding to avidin; a protease binding to a protease inhibitor; a nucleotide-binding protein binding to triazine; and an src kinase binding to casein or tyrosine. An example of an immunoglobulin G derivative includes an antibody fragment such as Fab.

3. Step to React the Ligand with the Insoluble Base Material

In this step, a ligand having a specific affinity for a target compound and having an amino group is mixed with the formyl group-containing insoluble base material to form an imine. More specifically, the formyl group of the insoluble base material and the amino group of the ligand are reacted to form an imino group.

A pH value of a reaction mixture for the imination reaction between the ligand and the insoluble base material may be 7.0 or more and less than 13.0, since an amount of the immobilized amino group-containing ligand and/or immobilization ratio may become larger in the range.

A solvent for the above-described imination reaction may be a buffer solution in terms of pH stability. The buffer solution usable in one or more embodiments of the present disclosure is not particularly restricted, and a conventionally publicly known buffer solution may be preferably used.

A temperature of the above-described imination reaction may be appropriately adjusted and may be −10° C. or higher and 50° C. or lower. The reaction temperature may be −10° C. or higher in terms of ability for the reaction mixture to flow. When the reaction temperature may be 50° C. or lower, the ligand and the formyl group of the insoluble base material are hardly inactivated. The reaction temperature is more preferably −5° C. or higher, even more preferably 0° C. or higher, and more preferably 45° C. or lower, even more preferably 40° C. or lower.

The reaction may be sustained until the ligand and the insoluble base material are sufficiently reacted. The reaction time may be specifically determined by a preparatory experiment or the like and may be, for example, 1 hour or more and 50 hours or less.

After the reaction, the reaction mixture may be subjected to an after-treatment by an ordinary method but may be used in the next step as it is, since an imino group is relatively unstable.

4. Step to Reduce Imino Group

In this step, the imino group formed in the previous step between the amino group of the ligand and the formyl group of the insoluble base material is reduced. Not only the imino group formed between the amino group of the ligand and the formyl group of the insoluble base material but also an unreacted excess formyl group can be sufficiently reduced in this step by using two or more kinds of reducing agents so that the ligand is immobilized on the insoluble base material more surely and an excess formyl group can be reduced. It is considered that, as a result, a leakage of the ligand can be remarkably reduced and non-specific adsorption due to an excess formyl group can be reduced. In addition, the above effects are exhibited by a small amount of reducing agents. One or more embodiments of the present disclosure may be industrially excellent, as the cost and environmental load can be reduced.

As described above, a leaked ligand amount can be remarkably reduced by using two or more kinds of reducing agents. More specifically, a leaked ligand amount can be reduced to 200 ng/mL or less in the condition of the Examples described below. The leaked ligand amount may be 150 ng/mL or less and even more preferably 100 ng/mL or less.

If a formyl group remains on the insoluble base material after the immobilization of the ligand, a compound other than a target compound may be non-specifically reacted with or adsorbed on the formyl group. As a result, only a target compound may not be selectively adsorbed. An amount of an excess formyl group per 1 mL of the insoluble base material may be 8 μmol or less, more preferably 5 μmol or less, and even more preferably 3 μmol or less.

The present inventors experimentally found that not only a leaked ligand amount can be reduced but also an excess formyl group can be reduced by using two or more kinds of reducing agents in the step to reduce the imino group. The present inventors also experimentally found that the above effect is further enhanced amazingly by separately and sequentially adding each reducing agent in comparison with the case where two or more kinds of the above reducing agents are concurrently used in combination.

The reducing agent usable in one or more embodiments of the present disclosure is not particularly restricted, and for example, a borane complex can be used. More specifically, an example of the reducing agent includes 4-(dimethylamino)pyridine borane, N-ethyldiisopropylamine borane, N-ethylmorpholine borane, N-methylmorpholine borane, N-phenylmorpholine borane, lutidine borane, triethylamine borane or trimethylamine borane, 4-(dimethylamine)pyridine borane, N-ethyldiisopropylamine borane, N-ethylmorpholine borane, N-methylmorpholine borane, N-phenylmorpholine borane, lutidine borane, ammonia borane, dimethylamine borane, pyridine borane, 2-methylpyridine borane (α-picoline borane), 3-methylpyridine borane (β-picoline borane), 4-methylpyridine borane (γ-picoline borane), N′N-diethylaniline borane, N′N-diisopropylethylamine borane, 2,6-lutidine borane, borane amine, trisdimethylamine borane, trismethylamine borane, borazine, 1,3,5-trimethylborazine, 2,4,6-trimethylborazine, hexamethylborazine, sodium cyanoborohydride and sodium triacetoxyborohydride.

An amount of the leaked ligand can be efficiently reduced by using a borane complex reducing agent containing a Lewis base ligand having a pKa of 6.5 or less among borane complex reducing agents. A pKa value of Lewis base in the borane complex containing a Lewis base ligand may be 6.4 or less, more preferably 6.3 or less, and even more preferably 6.2 or less. On the one hand, a lower limit of the pKa value is not particularly restricted. When a borane complex containing Lewis base having a lower pKa value is used, an amount of the ligand leaked from the adsorbent may be reduced. On the one hand, when the pKa value is excessively low, a complex with borane may not be possibly formed. Therefore, the pKa value may be 0.2 or more, more preferably not less than 0.5 or not less than 1.0, even more preferably not less than 2.0, not less than 3.0 or not less than 4.0, and even more preferably 5.0 or more.

A Lewis base having a pKa value of 6.5 or less usable in one or more embodiments of the present disclosure means a compound which can donate an electron pair to a borane to form a complex and which can exert a reduction action. An example of such a Lewis base includes an amine, a phosphine, a phenol, an amide, a urea and an oxime.

When an unshared electron pair of a nitrogen atom is conjugated to an aromatic ring, a pKa value may be decreased. An example of a Lewis base having a pKa value of 6.5 or less usable in one or more embodiments of the present disclosure includes a nitrogen-containing heteroaryl compound and/or an aromatic hydrocarbon compound having an amino group as a substituent.

The “nitrogen-containing heteroaryl compound” in this disclosure means an aromatic compound which contains at least one nitrogen atom in the aromatic ring and of which pKa value is 6.5 or less, and is exemplified by a 5-membered nitrogen-containing heteroaryl compound such as pyrrole; a 6-membered nitrogen-containing heteroaryl compound such as pyridine, pyridazine, pyrimidine and pyrazine; and a condensed nitrogen-containing heteroaryl compound such as quinoline, isoquinoline, phthalazine, quinazoline and quinoxaline.

The “aromatic hydrocarbon compound having an amino group as a substituent” means an aromatic hydrocarbon compound in which one or more amino groups as a substituent group are directly bound to the aromatic ring and of which pKa value is 6.5 or less. An example of the amino group includes —NH₂, a mono(C₁₋₆ alkyl)amino group and a di(C₁₋₆ alkyl)amino group. When the number of the amino group as a substituent group is larger, the pKa value tends to be larger; therefore, the number may be 1 or 2. An example of the aromatic hydrocarbon compound includes a C₆₋₁₂ aromatic hydrocarbon compound such as benzene, naphthalene and biphenyl.

The nitrogen-containing heteroaryl compound may have a substituent group such as an amino group as long as the pKa value thereof is 6.5 or less, and the aromatic hydrocarbon compound may have a substituent group other than an amino group as long as the pKa value thereof is 6.5 or less. An example of the substituent group other than an amino group includes one or more substituent groups selected from the group consisting of a C₁₋₆ alkyl group, a C₁₋₆ alkoxy group, a hydroxy group, a halogeno group, a cyano group and a nitro group.

In practice, since a pKa value is changed depending on the kind and the number of a substituent group, a Lewis base having a pKa value of 6.5 or less may be selected in reference to a document disclosing a pKa value or an actual measured value. The nitrogen-containing heteroaryl compound and the aromatic hydrocarbon compound are exemplified by pyridine; picoline such as α-picoline, β-picoline and γ-picoline; diphenylamine; toluidine such as o-toluidine, m-toluidine and p-toluidine; and pyrrole, but are not restricted thereto.

In addition, the pKa value of some aliphatic amines may be 6.5 or less depending on the kind and the number of a substituent group. An example of an aliphatic amine having a pKa value of 6.5 or less includes hydroxylamine or an alkoxyamine, such as hydroxylamine, methoxyamine, N-methylhydroxylamine and N,O-dimethylhydroxylamine; and a cyano C₁₋₆ alkylamine such as cyanomethyldiethylamine, di(cyanomethyl)amine and di(cyanoethyl)amine.

An example of a phosphine having a pKa value of 6.5 or less includes a tertiary phosphine having an electron withdrawing group, a secondary phosphine and a primary phosphine. An example of the tertiary phosphine having an electron withdrawing group includes 2-cyanoethyldi(C₁₋₆ alkyl)phosphine, phenyldi(C₁₋₆ alkyl)phosphine, di(2-cyanoethyl)C₁₋₆ alkylphosphine, triphenylphosphine and tri(2-cyanoethyl)phosphine. An example of the secondary phosphine includes di(C₁₋₆ alkyl)phosphine, diphenylphosphine and di(2-cyanoethyl)phosphine. An example of the primary phosphine includes C₁₋₆ alkylphosphine.

An example of a phenol having a pKa value of 6.5 or less includes a phenol having an electron withdrawing substituent group at o-position or p-position. For example, 2,4-dinitrophenol, 2-chrolophenol, 2-bromophenol and 4-nitrophenol can be used.

An example of an amide having a pKa value of 6.5 or less includes cyanamide, C₁₋₆ alkylcyanamide and acetamide.

An example of a urea having a pKa value of 6.5 or less includes urea, nitrourea and thiourea.

An example of an oxime having a pKa value of 6.5 or less includes oxamide oxime, benzamide oxime, α-phenylacetamide oxime, succinamide oxime and toluamide oxime.

The borane complex can be generally produced by reacting diborane produced using sodium borohydride with a Lewis base ligand.

An embodiment to add two or more kinds of reducing agents to a reaction mixture containing the imine is not particularly restricted. Two or more kinds of reducing agents may be simultaneously added, and it is preferred that two or more kinds of reducing agents are added separately and sequentially. When reducing agents are added separately and sequentially, an addition order is not particularly restricted. For example, an example of a reducing agent to be used first includes a borane complex reducing agent containing a Lewis base ligand having a pKa value of 6.5 or less. As such a borane complex reducing agent containing a Lewis base ligand having a pKa value of 6.5 or less, the above-described borane complex reducing agent may be used. An example of such a borane complex reducing agent includes pyridine borane and 2-methylpyridine borane. The other reducing agent is not particularly restricted and may be exemplified by dimethylamine borane, sodium triacetoxyborohydride and sodium cyanotrihydridoborate. The number of the reducing agent to be used may be 5 or less, more preferably not more than 4 or not more than 3, and may be 2.

When two or more kinds of reducing agents are added separately and sequentially, one kind of reducing agent is added and then other reducing agent may be immediately added, and it is preferred that one kind of reducing agent is added and then other reducing agent is added with a time interval. The time interval may be adjusted to 10 minutes or more and 24 hours or less. The reaction mixture may be left still between the additions of two or more kinds of reducing agents and may be stirred.

A solvent for the reduction reaction may be an aqueous solvent. An example of the aqueous solvent includes water; an aqueous solution such as a buffer solution; a water-miscible organic solvent; and a mixed solvent of an aqueous solution and a water-miscible organic solvent. The water-miscible organic solvent means an organic solvent which can be mixed with water without limit and is exemplified by a lower alcohol solvent such as methanol, ethanol and isopropanol; an amide solvent such as dimethylformamide and dimethylacetamide; and a sulfoxide solvent such as dimethylsulfoxide.

It is preferred to use the aqueous solvent in the reduction reaction of the present step, since a denaturation and a deterioration of the immobilized ligand can be inhibited in comparison with the case where an organic solvent is used. Since some of the amine-borane complex is insoluble in water, an appropriate amount of a water-miscible organic solvent may be added depending on a water solubility of the amine-borane complex to be used. A concentration of a water-miscible organic solvent in an aqueous solvent may be, for example, 70 mass % or less and more preferably 50 mass % or less. In order to dissolve the amine-borane complex, the concentration may be 2 mass % or more and more preferably 5 mass % or more.

The pH of a reaction mixture of the reduction reaction in the present step may be 2 or more and less than 12. When the pH is 2 or more, a decomposition of the imino group and a deactivation of the borane complex due to a reaction with water may be inhibited more surely. On the one hand, when the pH is less than 12, the reaction by the borane complex may be further accelerated. The pH may be 3 or more, and more preferably less than 10, and even more preferably less than 9.

Any reaction conditions such as a reaction temperature and a reaction time may be applied as long as the imino group can be sufficiently reduced, and the condition may be specifically determined by a preparatory experiment or the like. For example, a reaction time may be adjusted to 1 hour or more and 50 hours or less, and a reaction temperature may be adjusted to 0° C. or higher and 50° C. or lower.

It is preferred to remove a reagent other than the ligand covalently bound to the insoluble base material according to one or more embodiments of the present disclosure method by washing the adsorbent after the reaction. A washing agent and a washing method are not particularly restricted, and it is preferred to flow or add and stir a solution containing at least one selected from water, acetic acid, an alcohol, various organic solvents, an aqueous solution having a pH of 2 to 13, sodium chloride, potassium chloride, sodium acetate, disodium hydrogenphosphate, sodium dihydrogenphosphate, a buffer solution, a surfactant, urea, guanidine, guanidine hydrochloride and other regenerants. In addition, it is preferred to wash the adsorbent two or more times using the same or different solutions, since an amount of a leaked ligand is further reduced.

An amount of the immobilized ligand on the ligand-immobilized base material, of one or more embodiments of the present disclosure, per 1 mL of the formyl group-containing insoluble base material may be 1 mg or more and 500 mg or less. When the amount of the immobilized ligand may be 1 mg or more per 1 mL of the formyl group-containing insoluble base material, an amount of an adsorbed target compound may become large. When the amount may be 500 mg or less, a production cost may be lowered. The amount of the immobilized ligand per 1 mL of the formyl group-containing insoluble base material is more preferably 2 mg or more, even more preferably 3 mg or more, even more preferably 4 mg or more, and more preferably 120 mg or less, even more preferably 60 mg or less, even more preferably 30 mg or less.

In addition, the amount of the immobilized ligand on the ligand-immobilized base material of one or more embodiments of the present disclosure per 1 mL of the formyl group-containing insoluble base material may be 0.01 μmol or more and 15 μmol or less. When the amount of the immobilized ligand per 1 mL of the formyl group-containing insoluble base material may be 0.01 μmol or more, an amount of an adsorbed target compound may become large. When the amount may be 15 μmol or less, a production cost may be lowered. The amount of the immobilized ligand may be 0.03 μmol or more, even more preferably 0.05 μmol or more, even more preferably 0.1 μmol or more, and more preferably 5 μmol or less, even more preferably 2 μmol or less, even more preferably 1 μmol or less.

The amount of the immobilized ligand can be determined by measuring absorbances derived from the ligand in the reaction mixture supernatant before and after the immobilization reaction and calculating an amount of unreacted ligand on the basis of the difference of the measured absorbance values under presumption that all of the rest of the ligands bind to the insoluble base material. The amount of the immobilized ligand can be also determined by elemental analysis method. For example, the amount of the immobilized ligand can be determined by analyzing an N content in the ligand-immobilized base material in case of the amino group-containing ligand.

5. Usage Example of Adsorbent

When the adsorbent produced by strongly immobilizing the ligand on the insoluble base material in accordance with one or more embodiments of the present disclosure method is used for purifying a target compound, the ligand is remarkably prevented from being mixed with the target compound, since a leakage of the ligand is remarkably inhibited. In addition, when the adsorbent is used for purifying a target compound, a non-specific adsorption amount is expected to be reduced, since an excess formyl group is sufficiently inactivated.

In order to purify a target compound by using the adsorbent of one or more embodiments of the present disclosure, the target compound is selectively adsorbed on the adsorbent by contacting the adsorbent with a mixed liquid containing the target compound. A contacting method is not particularly restricted, and for example, the adsorbent may be added to the mixed liquid to be mixed and it is efficient and convenient that a column is filled with the adsorbent and the mixed liquid is flowed through the column.

For example, a column having a diameter of 0.1 cm or more and 2000 cm or less and height of 1 cm or more and 5000 cm or less may be used. When the diameter is 0.1 cm or more and the height is 1 cm or more, a target compound can be efficiently adsorbed. In addition, the diameter may be 2000 cm or less and the height may be 5000 cm or less in terms of an accuracy and an efficiency of the adsorption.

A residence time to contact the adsorbent with the mixed liquid containing a target compound may be 1 minute or more in terms of an accuracy of the adsorption and a durability of a device. On the one hand, the residence time may be 12 minutes or less in terms of efficiency. The residence time may be 2 minutes or more, even more preferably 3 minutes or more, and more preferably 10 minutes or less, even more preferably 9 minutes or less.

A specific adsorbing condition may be adjusted so that an amount of an adsorbed target compound per 1 mL of the adsorbent becomes 1 mg or more. When the adsorption amount is 1 mg or more, the purification can be efficiently performed. On the one hand, when the adsorption amount is 200 mg or less, an adsorbed target compound can be easily eluted from the adsorbent. The adsorption amount may be 10 mg or more and 150 mg or less, even more preferably 20 mg or more and 130 mg or less, and even more preferably 30 mg or more and 100 mg or less.

An amount of an adsorbed target compound can be determined as a static adsorption amount and a dynamic adsorption amount, and is not restricted thereto. For example, a static adsorption amount can be determined by sufficiently washing 0.5 mL of the adsorbent with a phosphate buffer having pH of 7.4, dissolving 70 mg of a target compound in 35 mL of the same phosphate buffer having pH of 7.4, contacting the solution with the washed adsorbent, stirring the mixture at 25° C. for 2 hours, and then measuring an amount of the reduced target amount in the supernatant.

After adsorbing a target compound on the adsorbent of one or more of the present disclosure, the adsorbent may be washed to remove a substance non-specifically adsorbed on the adsorbent. A condition of the washing is not particularly restricted, it is preferred that the adsorbent is sufficiently washed with a buffer solution having pH of 6.0 or more and 8.0 or less, ultrapure water, pure water, reverse osmosis water, distilled water or the like so that an adsorbed target compound is not desorbed.

Then, a purified target compound can be obtained by desorbing a target compound adsorbed on the adsorbent. In order to desorb a target compound from the adsorbent, for example, the adsorbent may be washed with a buffer solution having pH of 3.0 or more and 5.0 or less.

The present application claims the benefit of the priority date of Japanese patent application No. 2018-3100 filed on Jan. 12, 2018. All of the contents of the Japanese patent application No. 2018-3100 filed on Jan. 12, 2018, are incorporated by reference herein.

### EXAMPLES

Hereinafter, one or more embodiments of the present disclosure is described in more detail with Examples. The embodiments are, however, not restricted to the following Examples in any way, and it is possible to work one or more embodiments of the present disclosure according to the Examples with an additional appropriate change within the range of the above descriptions and the following descriptions. Such a changed embodiment is also included in the technical scope of the present disclosure.

### Example 1: Production of Adsorbent

A crosslinked cellulose particle, specifically a gel produced by the method described in JP 2009-242770 A, was used as an insoluble base material. Using 0.01 M citrate buffer of pH 3.4 produced by using trisodium citrate dihydrate manufactured by Satuma Kako Co., Ltd., citric acid monohydrate manufactured by Satuma Kako Co., Ltd. and reverse osmosis water, 70 mL of the insoluble base material was sufficiently washed on a glass filter. Then, the washed insoluble base material was added into a centrifuge tube, and the citrate buffer was added thereto to adjust the total liquid volume to 108 mL. An aqueous solution produced by dissolving 0.45 g of sodium periodate manufactured by Kishida Chemical Co., Ltd. in 17.6 mL of reverse osmosis water was added thereto, and the mixture was stirred at 6° C. for 40 minutes to oxidize the 1,2-diol group of the insoluble base material to a formyl group. A formyl group-containing insoluble base material was obtained by filtering the mixture with a glass filter and washing with a sufficient amount of reverse osmosis water.

Using 0.9 M dipotassium hydrogenphosphate aqueous solution produced from dipotassium hydrogenphosphate manufactured by Yoneyama Kagaku Kogyo Kaisha, Ltd. and reverse osmosis water, 15 mL of the obtained formyl group-containing insoluble base material was sufficiently washed on a glass filter. Then, the washed formyl group-containing insoluble base material was added into a separable flask, and the dipotassium hydrogenphosphate aqueous solution was added thereto to adjust the total liquid volume to 19 mL. Separately, Protein A having the amino acid sequence of SEQ ID NO: 2 described in WO 2011/118699 was produced by reference to the international publication. To the above formyl group-containing insoluble base material, 2.6 mL of 58 g/L Protein A aqueous solution was added. Then, the pH of the mixture was adjusted to 11 using 2 M of sodium hydroxide aqueous solution prepared from 24% sodium hydroxide aqueous solution manufactured by Kaname Chemicals Co., Ltd. and reverse osmosis water, and the mixture was stirred at 7° C. for 15 hours. Next, the total liquid volume was adjusted to 19 mL by taking out the supernatant, and a solution prepared by dissolving 48 mg (0.45 mmol) of α-picoline borane manufactured by Junsei Chemical Co., Ltd. in 3.2 mL of ethanol manufactured by Wako Pure Chemical Corporation and an aqueous solution prepared by dissolving 0.24 g (4.05 mmol) of dimethylamine borane in 2.2 mL of reverse osmosis water were simultaneously added thereto. Then, the pH of the mixture was adjusted to 7.6 by adding 2.4 M of citric acid aqueous solution prepared from citric acid mono hydrate and reverse osmosis water, the temperature was increased to 25° C., and the mixture was stirred for 4 hours.

The obtained base material was sequentially washed with reverse osmosis water, 0.1 M citric aqueous solution, a mixed aqueous solution of 0.05 M sodium hydroxide+0.5 M sodium sulfate manufactured by Ishida Chemicals Co., Ltd. and a citrate buffer solution of 0.5 M trisodium citrate dihydrate+citric acid monohydrate (pH=6) on a glass filter. Finally, the base material was washed with reverse osmosis water until an electric conductivity of the filtrate became 5 μS/cm or less to obtain a ligand-immobilized adsorbent.

### Comparative Example 1: Production of Adsorbent

A ligand-immobilized adsorbent was obtained similarly to Example 1 except that only 10 times the amount of an α-picoline borane solution was used without using dimethylamine borane.

### Comparative Example 2: Production of Adsorbent

A ligand-immobilized adsorbent was obtained similarly to Example 1 except that only 1.1 times the amount of a dimethylamine borane aqueous solution was used without using α-picoline borane.

### Example 2: Production of Adsorbent

A ligand-immobilized adsorbent was obtained similarly to Example 1 except that after the α-picoline borane was added and the pH of the mixture was adjusted to 7.6 by adding 2.4 M of citric acid aqueous solution, the temperature of the mixture was increased to 25° C., the mixture was stirred for 1 hour, dimethylamine borane aqueous solution was added thereto, and the mixture was stirred for 3 hours.

### Example 3: Production of Adsorbent

A ligand-immobilized adsorbent was obtained similarly to Example 2 except that the α-picoline borane added first was exchanged for pyridine borane (0.45 mmol).

### Example 4: Production of Adsorbent

In the procedures of Example 1, an ethanol solution (1.4 mL) of pyridine borane (0.45 mmol) was added to the reaction mixture (19 mL) of Protein A and the formyl group-containing insoluble base material. Then, the pH of the reaction mixture was adjusted to 7.6 by adding 2.4 M of citric acid aqueous solution, and then the temperature of the reaction mixture was increased to 25° C. After the reaction mixture was stirred for 1 hour, an aqueous solution of dimethylamine borane (3.6 mmol) was added thereto. After the reaction mixture was stirred for 3 hours, an ethanol solution of N′N-diethylaniline borane (0.45 mmol) was added thereto. The reaction mixture was stirred for 1 hour.

The obtained base material was sequentially washed with reverse osmosis water, 0.1 M citric acid aqueous solution, a mixed aqueous solution of 0.05 M sodium hydroxide+0.5 M sodium sulfate manufactured by Ishida Chemicals Co., Ltd., and a citrate buffer (0.5 M trisodium citrate dihydrate+citric acid monohydrate, pH=6) on a glass filter. Finally, the base material was washed with reverse osmosis water until an electric conductivity of the filtrate became 5 pS/cm or less to obtain a ligand-immobilized adsorbent.

### Test Example 1: Measurement of Excess Formyl Group Amount

An amount of a formyl group remaining on the insoluble base material was estimated from an amount of remaining phenylhydrazine after the reaction by using a fact that the excess formyl group is reacted with phenylhydrazine. Specifically, after 4 mL of each adsorbent was washed with 0.1 M sodium phosphate buffer of pH 8, the total liquid amount was adjusted to 6 mL, and 2 mL of 0.1 M sodium phosphate buffer of pH 8 in which phenylhydrazine was dissolved and added thereto. The mixture was stirred at 40° C. for 1 hour. An absorbance of an absorption maximum at about 278 nm of the reaction mixture supernatant was measured by UV measurement, and an excess formyl group amount was estimated on the basis of the consumed amount of phenylhydrazine calculated from the obtained amount of remaining phenylhydrazine in the supernatant. The result is shown in Table 1.

### Test Example 2: Measurement of Leaked Ligand Amount

An amount of a leaked ligand in the case where human IgG was adsorbed on the ligand-immobilized adsorbent prepared in the above Examples and Comparative examples was measured.

(1) Preparation of Solutions

The following Liquids A to E and Neutralizing liquid were prepared and defoamed before use.

Liquid A: PBS buffer solution of pH 7.4 prepared from Phosphate buffered saline manufactured by Wako Pure Chemical Corporation and reverse osmosis water.

Liquid B: Sodium acetate buffer solution prepared by adjusting the pH of 35 mM sodium acetate aqueous solution to 3.5 with acetic acid (the sodium acetate and acetic acid were manufactured by Wako Pure Chemical Corporation).

Liquid C: 0.1 M Phosphoric acid aqueous solution prepared from phosphoric acid manufactured by Wako Pure Chemical Corporation and reverse osmosis water.

Liquid D: 3 mg/mL IgG aqueous solution prepared from polyclonal antibody (“GAMMAGARD” manufactured by Baxter) and the above-described Liquid A.

Liquid E: 0.1 M Sodium hydroxide aqueous solution prepared from sodium hydroxide and reverse osmosis water.

Neutralizing liquid: 2 M Tris(hydroxymethyl)aminomethane aqueous solution prepared from tris(hydroxymethyl)aminomethane manufactured by Sigma-Aldrich and reverse osmosis water.

(2) Filling and Preparation

A column of diameter 0.5 cm×height 15 cm was filled with the adsorbent sample prepared in the above-described Examples and Comparative examples, and the column was connected to a column chromatography apparatus (“AKTAexplorer100” manufactured by GE Healthcare). Into collection tubes having a volume of 15 mL, 3 mL of Neutralizing liquid was preliminarily added, and the collection tubes were set on a fraction collector.

(3) Purification of IgG

Through the above-described column, 15 mL of Liquid A was flowed and then 50 mL of Liquid D, i.e. IgG aqueous solution, was flowed. Next, 21 mL of Liquid A was flowed and then 12 mL of Liquid B was flowed to elute IgG. Subsequently, 9 mL of Liquid C, 9 mL of Liquid A, 15 mL of Liquid E and 15 mL of Liquid A were flowed. The flow rate of Liquid D was adjusted to 0.5 mL/min and the flow rates of Liquids A, B, C and E were adjusted to 1 mL/min so that the residence time to contact the adsorbents became 6 minutes or 3 minutes.

(4) Measurement of Leaked Ligand Amount

An amount of the ligand contained in the IgG eluate was measured in order to determine the amount of the leaked ligand. Specifically, the eluate obtained in the above-described Test example 2(3) was taken, and IgG amount and ligand amount in the eluate were measured in order to determine a concentration of the ligand leaked in the purified IgG as a leaked amount. The ligand concentration was measured by ELISA method. Kinds and addition conditions of reducing agents and relationships between amounts of leaked ligand and excess formyl group are shown in Table 1.

TABLE 1

| | Reducing | Reducing | Reducing | Total used | | Amount of | Amount of |
|---|---|---|---|---|---|---|---|
| | agent 1 | agent 2 | agent 3 | amount of | | excess | leaked |
| | (Used | (Used | (Used | reducing | Addition | formyl group | ligand |
| | amount) | amount) | amount) | agent | method | mmol/L-r | ng/mL |
| Example 1 | α-Picoline borane | Dimethylamine borane | — | 4.5 mmol | Simultaneous | 2.2 | 153 |
| | (0.45 mmol) | (4.05 mmol) | | | addition | | |
| Comparative | α-Picoline borane | — | — | 4.5 mmol | — | 12.8 | 35 |
| example 1 | (4.5 mmol) | | | | | | |
| Comparative | — | Dimethylamine borane | — | 4.5 mmol | — | 2.4 | 409 |
| example 2 | | (4.5 mmol) | | | | | |
| Example 2 | α-Picoline borane | Dimethylamine borane | — | 4.5 mmol | Sequential | 2.1 | 18 |
| | (0.45 mmol) | (4.05 mmol) | | | addition | | |
| Example 3 | Pyridine borane | Dimethylamine borane | — | 4.5 mmol | Sequential | 2.3 | 4 |
| | (0.45 mmol) | (4.05 mmol) | | | addition | | |
| Example 4 | Pyridine borane | Dimethylamine borane | N′,N-Diethylaniline borane | 4.5 mmol | Sequential | 1.3 | 10 |
| | (0.45 mmol) | (3.6 mmol) | (0.45 mmol) | | addition | | |

As the results show in Table 1, when only one kind of reducing agent was used, there is room for improvement on a leaked ligand amount or excess formyl group amount; on the one hand, it was demonstrated that excess formyl group amount was sufficiently reduced and leaked ligand amount could be reduced by using two or more reducing agents. In addition, it was demonstrated that the effects became enhanced by separately and sequentially using two or more reducing agents rather than simultaneously using reducing agents.

1. A method for immobilizing a ligand having an amino group on a formyl group-containing insoluble base material, comprising the steps of:
mixing the ligand with the formyl group-containing insoluble base material to form an imine, and
reducing the imine by using two or more kinds of reducing agents.

2. The method according to claim 1, wherein the two or more kinds of reducing agents are separately added to reduce the imine.

3. The method according to claim 1, wherein the imine is reduced by using a borane complex as the reducing agent and then a different reducing agent, wherein the borane complex has a Lewis base ligand, and wherein the Lewis base ligand has a pKa that is 6.5 or less.

4. The method according to claim 3, wherein the Lewis base ligand having the pKa of 6.5 or less is a nitrogen-containing heteroaryl compound.

5. The method according to of claim 1, wherein the ligand is a peptide.

6. The method according to claim 5, wherein the peptide can specifically bind to an antibody.

7. The method according to claim 1, wherein the formyl group-containing insoluble base material is composed of at least one selected from the group consisting of a polysaccharide, a synthetic polymer and a glass.

8. The method according to claim 1, wherein a form of the formyl group-containing insoluble base material is at least one selected from the group consisting of a porous particle, a monolith and a porous membrane.

9. A method for purifying a target compound, comprising the steps of:
immobilizing the ligand on the formyl group-containing insoluble base material to produce an adsorbent by the method according to claim 1,
contacting a mixed liquid containing the target compound with the adsorbent to adsorb the target compound on the adsorbent, and
separating the target compound adsorbed on the adsorbent from the adsorbent.
